(19) United States
(12) Patent Application Publication
(10) Pub. No.: US 2026/0305732 A1
(43) Pub. Date: Oct. 8, 2026

(54) SOCK

(71) Applicant: RECOVERY CO., LTD., Kyoto-shi, Kyoto (JP)

(72) Inventor: Tamiro IKEMATSU, Kyoto-shi (JP)

(73) Assignee: RECOVERY CO., LTD., Kyoto-shi, Kyoto (JP)

(21) Appl. No.: 19/479,499

(22) PCT Filed: Nov. 28, 2024
(86) PCT No.: PCT/JP2024/042247

§ 371 Date: Oct. 28, 2025

(30) Foreign Application Priority Data
Dec. 4, 2023 (JP) 2023-204758

(51) Int. Cl. A41B 11/00

(52) U.S. Cl. CPC A41B 11/003; A41B 11/004

(57) ABSTRACT

A sock includes: a sock body; and an annular-shaped tightening portion provided in the sock body at a portion corresponding to at least one of bone head portions or basal portions of five metatarsals of a sock wearer so as to cover an instep and a sole, wherein a tightening force of the tightening portion is larger than a tightening force of a portion other than the tightening portion of the sock body, or a sock includes: a sock body; and an annular-shaped tightening portion provided in the sock body at a portion corresponding to at least one of a metatarsophalangeal joint, a Lisfranc joint, a Chopart joint, a toe joint, and a talocrural joint in a foot portion of a wearer, wherein a tightening force of the tightening portion is larger than a tightening force of a portion other than the tightening portion of the sock body.

### TECHNICAL FIELD

The present invention relates to a sock used for correcting a foot arch.

### BACKGROUND ART

FIG. 1 shows bones in a foot portion of a human, and FIG. 2 shows arch structure of the foot portion of a human. As shown in these figures, the foot portion (a portion from an ankle (or a malleolus) to a foot tip) of a human includes a total of 26 bones including 7 tarsals, 5 metatarsals, and 14 phalanges, and joints are respectively formed at articulations of bones. The 26 bones are connected to each other by ligaments, and bones, joints, ligaments, muscles, and the like are combined to form the arch structure.

The arch structure of the foot portion (hereinafter referred to as “foot arch”) includes a medial longitudinal arch connecting a calcaneus and a first metatarsal head, a lateral longitudinal arch connecting the calcaneus and a fifth metatarsal head, and a transverse arch connecting the first metatarsal head and the fifth metatarsal head. In Japan, the space formed in the sole by the foot arch is called “tsuchifumazu (arch of the foot)”.

The foot arch is supported by tissues such as ligaments, muscles, and tendons around the bones, and maintains an upright posture in cooperation with a series of muscles from the top of the head to the back, the buttocks, and the posterior portions of the thigh and the lower leg. In addition, the foot arch has a function as a cushion that absorbs every impact on the sole during biped walking.

The three-dimensional shape of the foot arch may be deformed due to a decrease in muscle strength of the sole from aging or a lack of exercise, an expansion of the ligament, or an increase in weight. When the three-dimensional shape of the foot arch deforms, the balance between the foot arch and each site from the top of the head to the back, the buttocks, and the posterior portions of the thigh and the lower leg is disturbed. As a result, the upright posture cannot be properly maintained, and a bad posture such as round shoulders is caused. In addition, in the condition where the foot arch deforms and there is no space of the sole (flat foot), the impact on the sole cannot be sufficiently absorbed during biped walking, and the knee and the waist suffer shocks.

Patent Literature 1, for example, discloses a sock in which an arch support portion is provided in a sock body at a portion corresponding to a foot arch so as to wind around the entire circumference of the sole and the instep. The arch support portion is formed by knitting a yarn that is less likely to stretch than a yarn for knitting the sock body into the ground weave of the sock body. With such structure, the arch support portion of the sock body has a stronger tightening force than other portions. Therefore, when a person having a flat sole due to collapse of the three-dimensional shape of the foot arch wears the sock having the above structure, the sole corresponding to the foot arch and the instep are tightened by the arch support portion, and the foot arch is corrected to a normal three-dimensional shape.

### CITATION LIST

### Patent Literature

Patent Literature 1: JP 2020-133046 A

### SUMMARY OF INVENTION

### Technical Problem

For example, in the case of a bow-legged person, the weight is applied to the lateral side (little toe side) of the foot portion, so that the foot arch may incline to the lateral side of the foot portion. Also in a case where the foot arch is inclined, the balance between the foot arch and each site from the top of the head to the back, the buttocks, and the posterior portions of the thigh and the lower leg collapses to cause a bad posture. However, in the sock described in Patent Literature 1, the foot arch in the inclined condition cannot be sufficiently corrected.

The problem to be solved by the present invention is to make it possible to correct the foot arch in various defective conditions to normal conditions by wearing the sock.

### Solution to Problem

A sock according to a first mode of the present invention made to solve the above problem includes:
a sock body; and
an annular-shaped tightening portion provided in the sock body at a portion corresponding to at least one of bone head portions or basal portions of five metatarsals of a wearer so as to cover an instep and a sole,
wherein a tightening force of the tightening portion is larger than a tightening force of a portion other than the tightening portion of the sock body.

In addition, a sock according to a second mode of the present invention made to solve the above problem includes:
a sock body; and
an annular-shaped tightening portion provided in the sock body at a portion corresponding to at least one of a metatarsophalangeal joint, a Lisfranc joint, a Chopart joint, a toe joint, and a talocrural joint, which are five types of joints in a foot portion of a wearer, wherein a tightening force of the tightening portion is larger than a tightening force of a portion other than the tightening portion of the sock body.

### ADVANTAGEOUS EFFECTS OF INVENTION

A metatarsophalangeal joint is located at the bone head portions of the five metatarsals, and a Lisfranc joint is located at the basal portions of the five metatarsals. Accordingly, a wearer of the sock of the first mode of the present invention is subjected to strong tightening by the tightening portion of the instep and the sole surrounding the metatarsophalangeal joint and/or the Lisfranc joint in a foot portion, and a foot arch is corrected to a three-dimensional shape. In addition, since the metatarsophalangeal joint and/or the Lisfranc joint in the foot portion of the wearer are stimulated by the tightening force of the tightening portion, in a case where the foot arch of the wearer is inclined or the positions of the bones in the foot portion are shifted, it is possible to return them to a normal condition.

In addition, the wearer of the sock of the second mode of the present invention is subjected to strong tightening by the tightening portion of the instep and the sole surrounding at least one of five types of joints in the foot portion, and a foot arch is corrected to a three-dimensional shape. In addition, since at least one of the five types of joints is stimulated by the tightening force of the tightening portion, in a case where the foot arch of the wearer is inclined or the positions of the bones in the foot portion are shifted, it is possible to return them to a normal condition.

### BRIEF DESCRIPTION OF DRAWINGS

FIG. 1 A view showing bones in a foot portion of a human.

FIG. 2 A view showing foot arches of a human.

FIG. 3 A view of the right foot on which a sock according to a first embodiment of the present invention is worn, as viewed from a big toe side.

FIG. 4 A view (a) as viewed from an instep side and a view (b) as viewed from a sole side of the right foot on which the sock according to the above embodiment is worn.

FIG. 5 A view of the right foot on which a sock according to a second embodiment of the present invention is worn, as viewed from the big toe side.

FIG. 6 A view (a) as viewed from the instep side and a view (b) as viewed from the sole side of the right foot on which the sock according to the above embodiment is worn.

FIG. 7 A view of the right foot on which a sock according to a third embodiment of the present invention is worn, as viewed from the big toe side.

FIG. 8 A view (a) as viewed from the instep side and a view (b) as viewed from the sole side of the right foot on which the sock according to the above embodiment is worn.

FIG. 9 A view of the right foot on which a sock according to a fourth embodiment of the present invention is worn, as viewed from the big toe side.

FIG. 10 A view (a) as viewed from the instep side and a view (b) as viewed from the sole side of the right foot on which the sock according to the above embodiment is worn.

FIG. 11 A view of the right foot on which a sock according to a fifth embodiment of the present invention is worn, as viewed from the big toe side.

FIG. 12 A view (a) as viewed from the instep side and a view (b) as viewed from the sole side of the right foot on which the sock according to the above embodiment is worn.

FIG. 13 A view of the right foot on which a sock according to a sixth embodiment of the present invention is worn, as viewed from the big toe side.

FIG. 14 A view (a) as viewed from the instep side and a view (b) as viewed from the sole side of the right foot on which the sock according to the above embodiment is worn.

FIG. 15 A view (a) as viewed from the instep side of the left foot on which a sock according to a reference example is worn and a view (b) as viewed from the instep side of the right foot on which the same sock is worn.

FIG. 16 Views showing examples of treatment.

### DESCRIPTION OF EMBODIMENTS

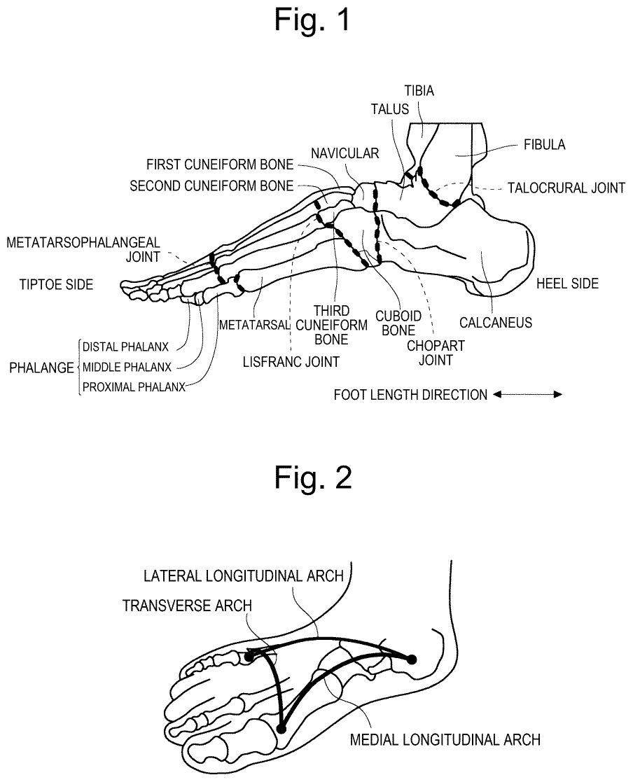

As described above, the foot portion includes a plurality of bones and joints at a plurality of portions (see FIG. 1). The condition of the joint affects the daily movement and walking of the human. In addition, as can be seen from the fact that the spinal cord reflex is observed in the foot portion, the condition of the bones in the foot portion and the condition of the bones of the back are said to be related. Specifically, the conditions of the calcaneus and the sacrum, the talus and the lumbar vertebra, the navicular and the metatarsal and the thoracic vertebra, and the phalange and the cervical vertebra are associated with each other.

When five representative joints (a metatarsophalangeal joint, a Lisfranc joint, a Chopart joint, a toe joint, and a talocrural joint) among the joints in the foot portion are stimulated, it is possible to place the bones in the foot portion at correct positions. It is known that when the bones in the foot portion are at the correct positions, the backbone is in the condition of maintaining a natural curve, and a stable posture in which a burden is not applied to the body, that is, a so-called neutral position (natural relaxation posture).

Therefore, the present inventor has conducted, for a plurality of subjects, an experiment of winding a tape around portions corresponding to the above-described five joints and observing the walking posture and the stationary posture of the subjects before and after winding the tape. In addition, hearing investigations from each subject were conducted on the conditions of the joints before winding the tape and the conditions of the joints after winding the tape. As a result, the present inventor has found that by winding a tape at portions respectively corresponding to the five joints, the conditions of the joints change, and the walking posture and the stationary posture change accordingly.

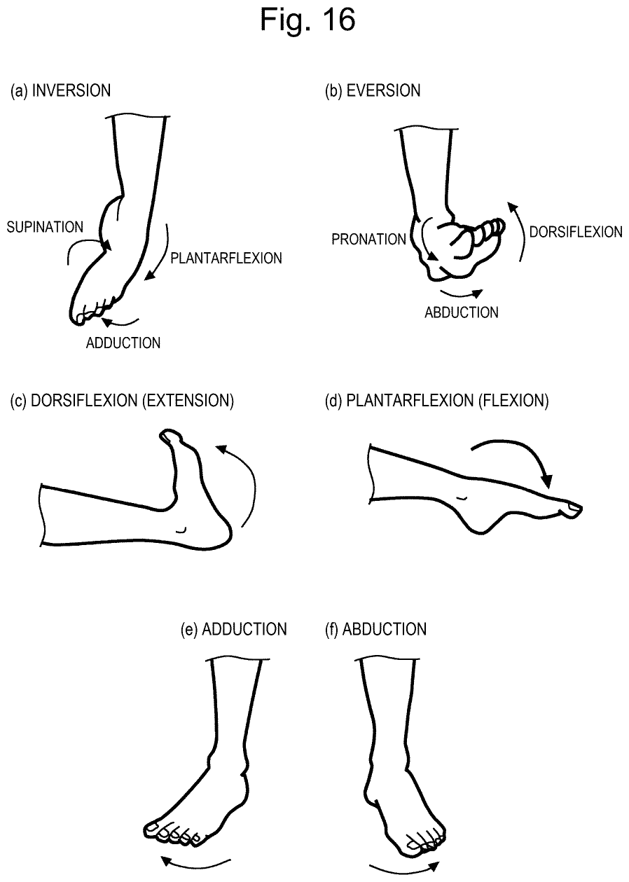

Such a change in the conditions of the joints and a change in the postures are considered to be caused by stimulation of the joints and the bones near the joints by winding a tape around the portions of the instep and the sole corresponding to the joints in the foot portion and tightening the instep and the sole. Stimulating the joints in the foot portion and the bones near the joints corresponds to, for example, treatments performed to improve the walking posture and the stationary posture of the subjects as shown in (a) to (f) of FIG. 16. Therefore, the present inventor has considered imparting an action corresponding to the above-described treatment to a wearer of a sock by wearing the sock, and has reached the present invention. The sock according to the present invention has been made based on knowledge and the like obtained from the above-described experiment and hearing investigation.

Hereinafter, embodiments for carrying out the present invention will be described with reference to the drawings.

### First Embodiment

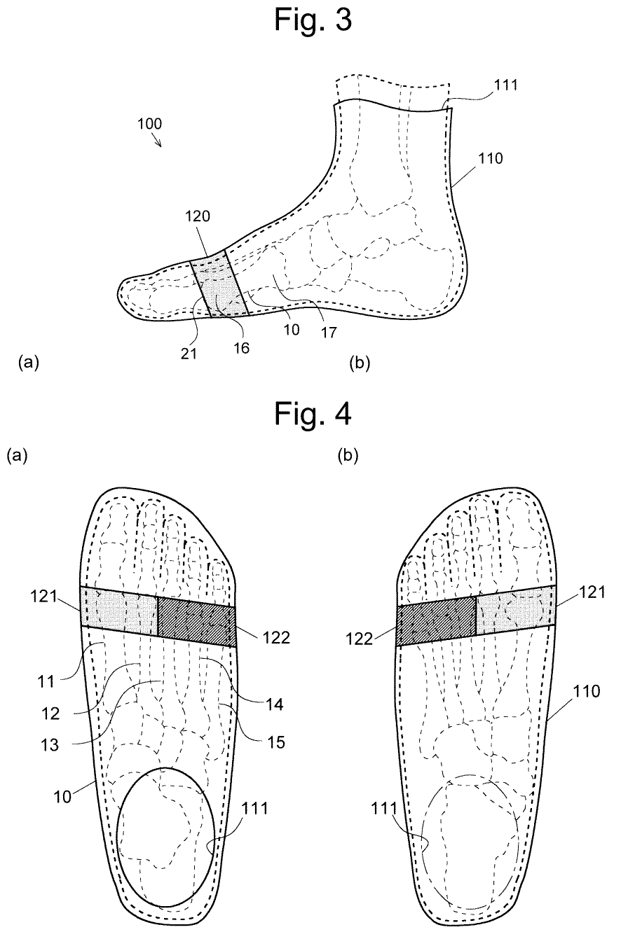

A first embodiment includes a pair of socks including a right foot sock and a left foot sock. FIG. 3 is a view of the right foot on which the right foot sock of the first embodiment is worn as viewed from the big toe side, FIG. 4(a) is a view of the right foot on which the right foot sock is worn as viewed from the instep side, and FIG. 4(b) is a view of the right foot on which the right foot sock 100 is worn as viewed from the sole side. Since the right foot sock and the left foot sock (not shown) have bilaterally symmetrical shapes and have the same structure and the same function, the structure, function, and the like of the socks will be described below without distinguishing between the right and left.

The sock 100 includes a sock body 110 having a foot insertion opening 111 and a tightening portion 120 formed in the sock body 110. The tightening portion 120 is formed in an annular shape so as to cover, from the instep to the sole, a portion where there are bone head portions 16 of five metatarsals B1 (a first metatarsal 11, a second metatarsal 12, a third metatarsal 13, a fourth metatarsal 14, and a fifth metatarsal 15) of the foot of the wearer. A portion where there are basal portions 17 of the metatarsals is not covered with the tightening portion 120. The tightening portion 120 is formed to have substantially the same width over the entire circumference, and has a width of 20 to 50 mm, for example.

The tightening portion 120 includes a medial tightening portion 121 located on the big toe side and a lateral tightening portion 122 located on the little toe side. The medial tightening portion 121 is formed to cover a portion corresponding to the first metatarsal 11 of the sole to a portion corresponding to the second metatarsal 12 of the instep. On the other hand, the lateral tightening portion 122 is formed to cover at least portions corresponding to the third metatarsal 13, the fourth metatarsal 14, and the fifth metatarsal 15 of the instep and the sole in the foot portion. It is sufficient that the medial tightening portion 121 covers at least a portion corresponding to the first metatarsal 11 of the instep and the sole in the foot portion.

The sock body 110 is made of a material such as a knitted fabric or a woven fabric having stretchability, and the tightening portion 120 of the sock body 110 is formed to have a lower stretch rate than other portions (non-tightening portions). The sock body 110 may be formed by joining the tightening portion 120 and the non-tightening portions made of another material, or may be formed by applying a process for reducing the stretch rate to a portion corresponding to the tightening portion 120 of the sock body 110. In either case, the non-tightening portions of the sock body 110 have the same degree of stretchability as that of a general sock, and the tightening portion 120 is formed to have lower stretchability than the non-tightening portions. In addition, the medial tightening portion 121 forming the tightening portion 120 is formed to have a lower stretch rate than the lateral tightening portion 122. With such structure, the wearer of the sock 100 is subjected to tightening of the portion covered with the tightening portion 120 of the sole and the instep with a stronger force than the other portions, and further subjected to tightening of the portion covered with the medial tightening portion 121 with a stronger force than the portion covered with the lateral tightening portion 122.

In a case where the sock body 110 is formed by joining the tightening portion 120 and the non-tightening portions, it is possible to form the tightening portion 120 by, for example, the following method.

In a first method, the tightening portion 120 is formed by joining the medial tightening portion 121 having predetermined stretchability and the lateral tightening portion 122 made of a highly stretchable material having higher stretchability than the medial tightening portion 121.

In a second method, two band-shaped materials whose stretch rate gradually increases from one end side toward the other end side are prepared, and the two band-shaped materials are joined at respective one end sides and the respective other end sides to form the tightening portion 120. In this case, the one end sides of the two joined materials correspond to the medial tightening portion 121, and the other end sides correspond to the lateral tightening portion 122.

In a third method, the tightening portion 120 is formed from a tubular knitted fabric or woven fabric in which stitches or weaves of a portion corresponding to the medial tightening portion 121 and a portion corresponding to the lateral tightening portion 122 are different. The tightening portion 120 having this structure has no seam.

In addition, examples of a processing method in the case of performing a process for lowering the stretch rate on a portion corresponding to the tightening portion 120 of the sock body 110 include, for example, the following method.

In a case where the sock body 110 is formed of a knitted fabric, a reinforcement yarn is knitted in a portion corresponding to the tightening portion 120, the stitch density (size of the stitch) at the time of knitting the knitted fabric is reduced, or a knitted pattern with low stretchability (tuck knitting, float knitting, and the like) is used.

In a case where the sock body 110 is formed of a woven fabric, the ratio of the elastic yarn forming the portion corresponding to the tightening portion 120 is increased, the elastic yarn is thickened, or an elastic yarn having a high elastic modulus is used.

In a case where the sock body 110 is formed of a knitted fabric or a woven fabric, a portion corresponding to the tightening portion 120 is coated with a resin such as silicone, or a fabric or material of low-stretchability such as a power net is stuck.

In a case where the sock 100 having the above structure is worn, the instep and the portion corresponding to the foot arch of the sole of the wearer are tightened by the tightening portion 120, so that the foot arch is corrected to a three-dimensional shape. In addition, a larger pressure is applied to a portion corresponding to the medial tightening portion 121 of the foot portion of the wearer than a portion corresponding to the lateral tightening portion 122. As a result, it is possible to obtain an action equivalent to that in a case where “eversion” of FIG. 16(b) or “abduction” of FIG. 16(f) is performed on the foot portion of the wearer.

Therefore, the foot arch of the foot portion in which the big toe side is slightly lifted due to the deviation of the center of gravity is pushed back to a normal position, and as a result, the foot arch is corrected to a normal condition. In the present embodiment, since the portion corresponding to the bone head portions 16 of the metatarsals (end portions on a metatarsophalangeal joint 21 side) is tightened by the tightening portion 120, it is possible to effectively improve the posture of a person with a rounded back who tends to concentrate his/her weight on the metatarsophalangeal joint 21.

### Second Embodiment

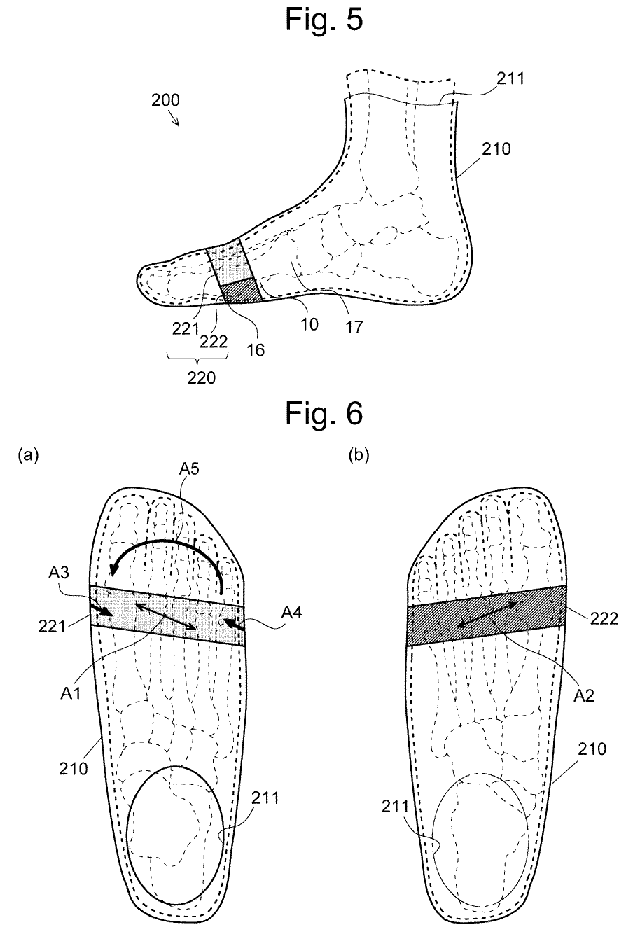

FIG. 5 is a view of the right foot on which a right foot sock of a second embodiment is worn as viewed from the big toe side, FIG. 6(a) is a view of the right foot on which the right foot sock is worn as viewed from the instep side, and FIG. 6(b) is a view of the right foot on which the right foot sock is worn as viewed from the sole side. Similarly to the first embodiment, since the socks according to the second embodiment include a right foot sock and a left foot sock having bilaterally symmetrical shapes, the structure, function, and the like of the socks will be described without distinguishing between the right and the left.

A sock 200 of the second embodiment includes a sock body 210 having a foot insertion opening 211 and a tightening portion 220 formed in the sock body 210. The tightening portion 220 includes an instep side tightening portion 221 covering the instep and a back side tightening portion 222 covering the sole. The instep side tightening portion 221 is formed such that a stretch rate in directions (directions indicated by an arrow Al in FIG. 6(a)) inclined toward the tiptoe side from the lateral side (little toe side) to the medial side (big toe side) is larger than stretch rates in other directions. Similarly, the back side tightening portion 222 is formed such that a stretch rate in directions (directions indicated by an arrow A2 in FIG. 6(b)) inclined toward the tiptoe side from the lateral side (little toe side) to the medial side (big toe side) is larger than stretch rates in other directions.

The position of the tightening portion 220 in the sock 200 of the present embodiment is the same as the position of the tightening portion 120 in the sock 100 of the first embodiment. In addition, the method of forming the sock body 210 and the tightening portion 220 is the same as the method of forming the sock body 110 and the tightening portion 120 of the first embodiment, and it is possible to use various methods as described above.

The wearer wearing the sock 200 according to the present embodiment is subjected to tightening of the instep and the portion corresponding to the foot arch of the sole by the tightening force of the tightening portion 220. Therefore, the foot arch of the wearer is corrected to a normal three-dimensional shape. In addition, the wearer of the sock 200 is subjected to application by the instep side tightening portion 221 of a force in a direction indicated by an arrow A3 in FIG. 6(a) to a big toe side end portion of the instep and a force in a direction indicated by an arrow A4 in FIG. 6(a) to the little toe side end portion. As a result, a force indicated by an arrow A5 in FIG. 6(b) is applied to the entire foot portion of the wearer. When such a force is applied to the foot portion, it is possible to obtain an effect equivalent to that in a case where the treatment of “inversion” shown in FIG. 16 is performed on the foot portion. As a result, for example, the joints hardened by, for example, injuries, sequelae of surgery, or aging are stimulated, and the foot portion, backbone, and entire body of the wearer recover to the neutral position.

### Third Embodiment

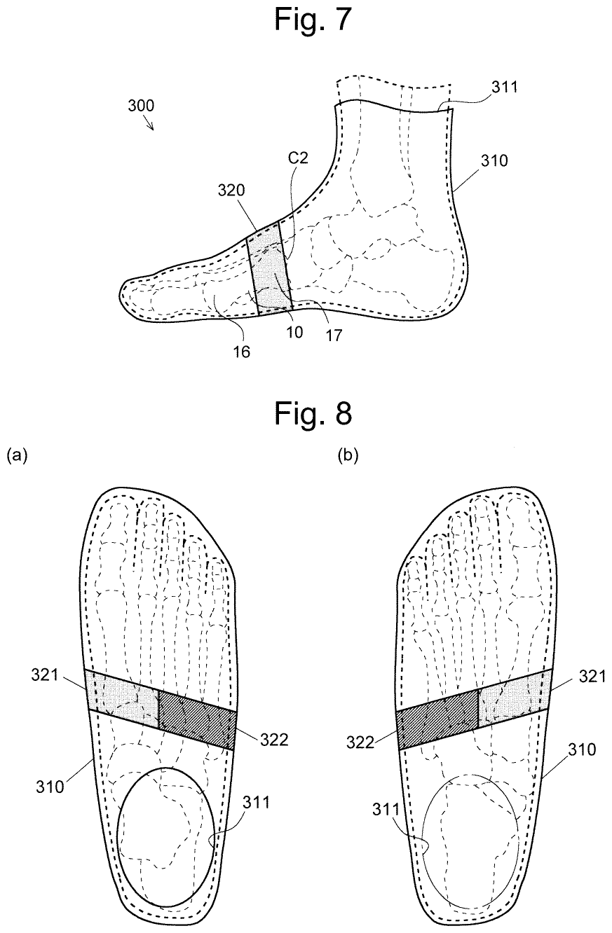

FIG. 7 is a view of the right foot on which a right foot sock of a third embodiment is worn as viewed from the big toe side, FIG. 8(a) is a view of the right foot on which the right foot sock is worn as viewed from the instep side, and FIG. 8(b) is a view of the right foot on which the right foot sock is worn as viewed from the sole side. Similarly to the first embodiment, since the socks according to the third embodiment include a right foot sock and a left foot sock having bilaterally symmetrical shapes, the structure, function, and the like of the socks will be described without distinguishing between the right and the left.

A sock 300 of the third embodiment includes a sock body 310 having a foot insertion opening 311 and a tightening portion 320 formed in the sock body 310. In the sock 300 of the present embodiment, the position of the tightening portion 320 is different from the position of the tightening portion 120 in the sock 100 of the first embodiment. That is, the tightening portion 320 is disposed in the sock body 310 so as to cover a portion corresponding to the basal portions 17 of the five metatarsals B1 from the instep to the sole and not to cover the bone head portions 16 of the metatarsals B1. Since the structure of the sock 300 other than the position of the tightening portion 320 is the same as the structure of the sock 100 of the first embodiment, a detailed description will be omitted.

The wearer wearing the sock 300 according to the present embodiment is subjected to tightening by the tightening portion 320 of the portion corresponding to the basal portions 17 of the metatarsals of the instep and the sole. Therefore, the foot arch is corrected to a three-dimensional shape. In addition, a larger pressure is applied to a portion corresponding to the medial tightening portion 321 of the foot portion of the wearer of the sock 300 than a portion corresponding to the lateral tightening portion 322. Therefore, in a case where the big toe side of the foot portion is slightly lifted due to the deviation of the center of gravity of the wearer, the bones in the foot portion are pushed back to normal positions, making it possible to correct the foot arch to a normal condition. In addition, in the present embodiment, since the portion corresponding to the basal portions 17 of the metatarsals (end portions on a Lisfranc joint 22 side) of the instep and the sole is tightened by the tightening portion 320, it is possible to effectively improve the posture of a person with a rounded lower back who tends to concentrate his/her weight on the Lisfranc joint 22.

### Fourth Embodiment

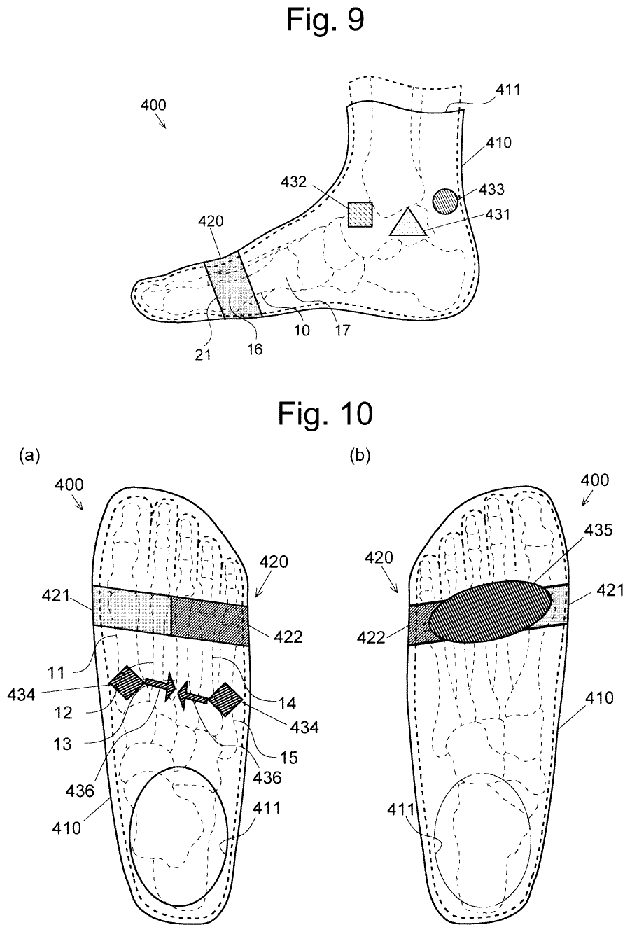

FIG. 9 is a view of the right foot on which a right foot sock of a fourth embodiment is worn as viewed from the big toe side, FIG. 10(a) is a view of the right foot on which the right foot sock is worn as viewed from the instep side, and FIG. 10(b) is a view of the right foot on which the right foot sock is worn as viewed from the sole side. Similarly to the first embodiment, since the socks according to the fourth embodiment include a right foot sock and a left foot sock having bilaterally symmetrical shapes, the structure, function, and the like of the socks will be described without distinguishing between the right and the left.

The sock 400 according to the fourth embodiment includes a sock body 410 having a foot insertion opening 411, a tightening portion 420 formed in the sock body, and a plurality of indications (marks) provided on the surface of the sock body 410. The sock 400 of the present embodiment is different from the sock 100 of the first embodiment in having indications, and is the same as the sock 100 of the first embodiment in the other structure. Hereinafter, the indications of the sock 400 will be described in detail, and the description of the structure of the sock 400 other than the indications will be omitted.

The indications are for indicating treatment sites effective for ameliorating the physical disorder caused as a result of the foot arch collapsing, and are respectively provided at positions corresponding to the treatment sites of the wearer on the surface of the sock body 410. Specifically, on the surface of the sock body 410, indications 431, 432, 433, 434, and 435 are provided at two sites respectively corresponding to the left and right malleoluses (ankle joint malleoluses) of the wearer, two sites closer to the toe side than the malleoluses, two sites closer to the heel side than the malleoluses, sites respectively corresponding to the first metatarsal 11 and the fifth metatarsal 15 of the instep, and the surface of the tightening portion 420 located on the sole, respectively. In addition, between the two indications 434, indications 436 of a pair of arrows disposed such that the arrow heads face each other are attached. The indications 436 of arrows are disposed on a straight line connecting the two indications 434. In the present embodiment, the indications 431, 432, 433, 434, and 435 have a triangular shape, a quadrangular shape, a circular shape, a rhombic shape, and an elliptical shape, respectively, but may have any shape, and may all have the same shape.

The above indications 431 to 436 are formed by coating the surface of the sock body 410 with a urethane resin or attaching a thin resin sheet. In the case of this structure, since it is possible to distinguish the sock body 410 and the indications 431 to 436 from each other by the different tactile sensation, the color of the sock body 410 and the color of the indications 431 to 436 may be the same or different.

In addition, the indications 431 to 436 may be made by making the color of the knitted fabric or the woven fabric forming the portions corresponding to the indications 431 to 436 of the sock body 410 different from the other portions. In the case of this structure, it is possible to eliminate the sense of discomfort caused by providing the indications 431 to 436.

The above indications 431 to 435 indicate treatment sites on the foot portion of the wearer of the sock 400, and the indications 436 indicate directions in which a force is applied when the sites indicated by the indications 434 are subjected to treatment. In addition, the respective sizes of the indications 431 to 435 indicate regions subjected to treatment, and usually, portions corresponding to the small indications 431 to 434 are subjected to treatment of pressing with a finger (finger pressing) and a portion corresponding to the large indication 435 is subjected to treatment of pressing with a palm. In addition, the indications 436 respectively indicate directions in which the indications 434 are pressed.

Therefore, the practitioner can perform appropriate treatment on appropriate sites in the foot portion by pushing the foot portion with his/her fingers or palm against the wearer of the sock 400 from above the indications 431 to 435. Thus, in addition to the effect of correcting the shape of the foot arch of the wearer of the sock 400, the sock 400 has an effect of assisting the treatment for alleviating the physical disorder due to the collapse of the foot arch.

The indications 431 to 436 may be provided with, for example, information such as a number indicating the order of treatment and the name of the treatment site. In addition, the sock body 410 does not need to be provided with all the six types of indications 431 to 436, and may be provided with one to five types selected from the six types of indications 431 to 436. Furthermore, the indications may be provided at positions other than the positions shown in FIG. 9 and FIG. 10. In addition, the indications 431 to 436 can be attached to the sock 200 of the second embodiment.

### Fifth Embodiment

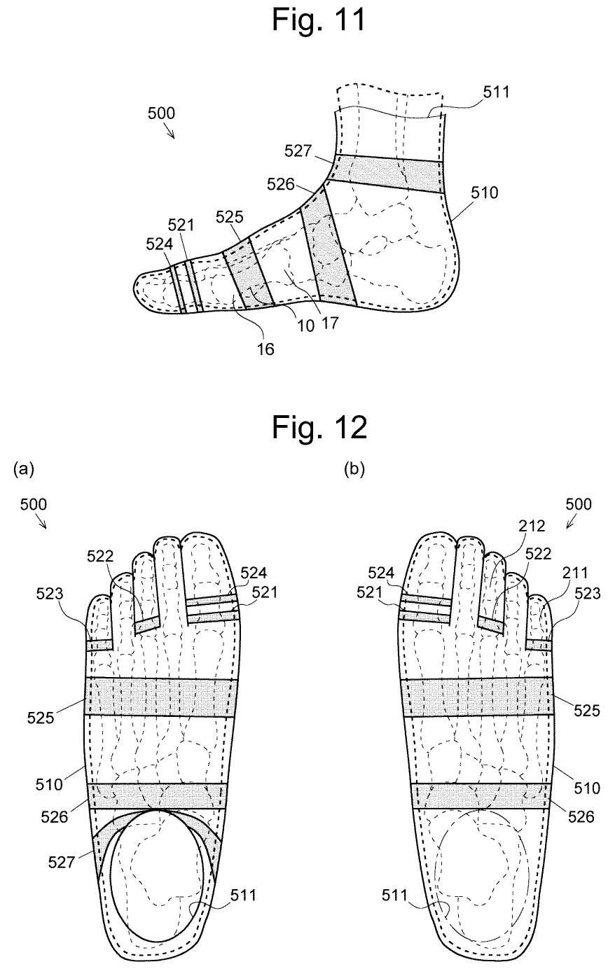

FIG. 11 is a view of the right foot on which a right foot sock of a fifth embodiment is worn as viewed from the big toe side, FIG. 12(a) is a view of the right foot portion on which the right foot sock is worn as viewed from the instep side, and FIG. 12(b) is a view of the right foot portion on which the right foot sock is worn as viewed from the sole side. Similarly to the first embodiment, since the socks according to the fifth embodiment include a right foot sock and a left foot sock having bilaterally symmetrical shapes, the structure, function, and the like of the socks will be described without distinguishing between the right and the left.

A sock 500 of the fifth embodiment includes a sock body 510 having a foot insertion opening 511 and a plurality of tightening portions formed in the sock body 510. The sock 500 of the present embodiment is a five-toe type sock in which the portion covering the toe is separated into five portions.

The tightening portions of the sock 500 of the present embodiment include a big toe tightening portion 521, a middle toe tightening portion 522, and a little toe tightening portion 523, which are respectively provided in regions corresponding to proximal end portions (portions close to the metatarsophalangeal joint) of a first proximal phalanx (big toe), a third proximal phalanx (middle toe), and a fifth proximal phalanx (little toe) of the sock body 510, a big toe tip side tightening portion 524 provided in a region corresponding to a portion on a tiptoe side of the first proximal phalanx, a middle foot tightening portion 525 provided in a region corresponding to a space between the metatarsophalangeal joint and the Lisfranc joint, a central tightening portion 526 provided in a region corresponding to the Chopart joint, and one malleolus tightening portion 527 provided in a region corresponding to the talocrural joint (malleolus). Each of the seven tightening portions 521 to 527 has an annular shape and is formed to cover a region on the instep side and the sole side of the sock body 510.

The sock of the fifth embodiment is a male sock made in consideration of arch structure of a male foot portion. When a male wearer wears the sock, it is possible to more effectively correct the foot arch to a normal condition, and to correct the walking posture and the stationary posture to a normal condition.

### Sixth Embodiment

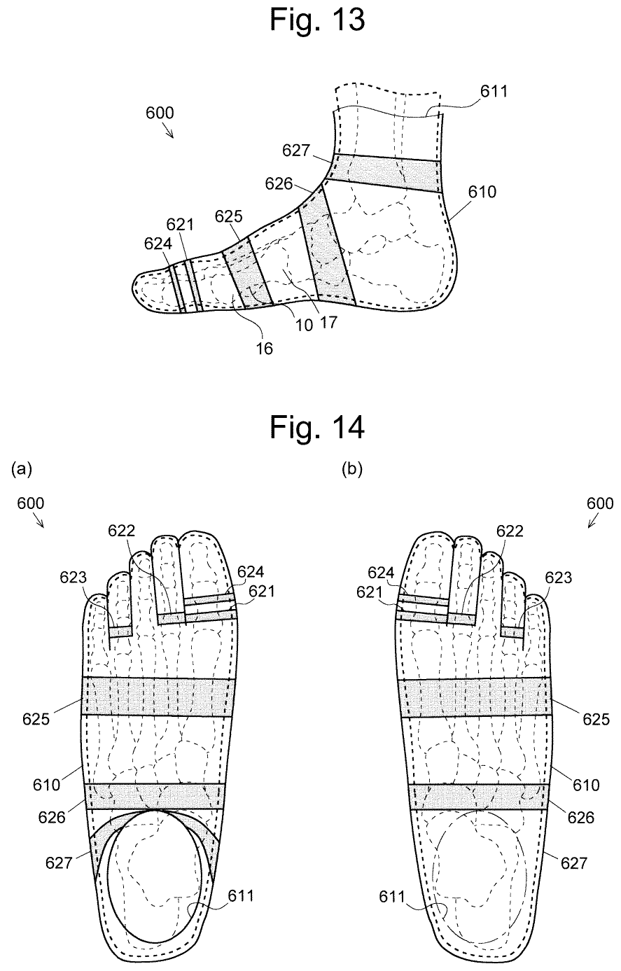

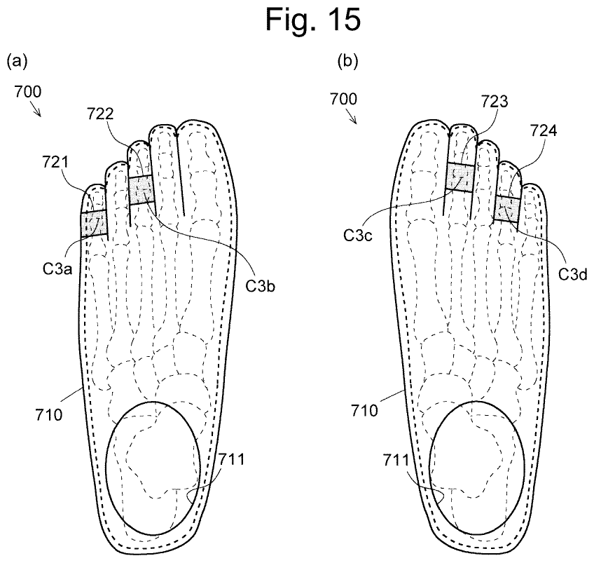

FIG. 13 is a view of the right foot on which a right foot sock of a sixth embodiment is worn as viewed from the big toe side, FIG. 14(a) is a view of the right foot portion on which the right foot sock is worn as viewed from the instep side, and FIG. 14(b) is a view of the right foot portion on which the right foot sock is worn as viewed from the sole side. Similarly to the first embodiment, since the socks according to the sixth embodiment include a right foot sock and a left foot sock having bilaterally symmetrical shapes, the structure, function, and the like of the socks will be described without distinguishing between the right and the left.

A sock 600 of the sixth embodiment includes a sock body 610 having a foot insertion opening 611 and a plurality of tightening portions formed in the sock body 610. Similarly to the sock 500 of the fifth embodiment, the sock 600 of the present embodiment is also a five-toe type sock in which the portion covering the toe is separated into five portions.

The tightening portions of the sock 600 of the present embodiment include a big toe tightening portion 621, a second toe tightening portion 622, and a fourth toe tightening portion 623, which are respectively provided in regions corresponding to proximal end portions (portions close to the metatarsophalangeal joint) of the first proximal phalanx (big toe), a second proximal phalanx (second toe), and a fourth proximal phalanx (fourth toe) of the sock body 610, a big toe tip side tightening portion 624 provided in a region corresponding to a portion on a tiptoe side of the first proximal phalanx, a middle foot tightening portion 625 provided in a region corresponding to a space between the metatarsophalangeal joint and the Lisfranc joint, a central tightening portion 626 provided in a region corresponding to the Chopart joint, and one malleolus tightening portion 627 provided in a region corresponding to the talocrural joint (malleolus). Each of the seven tightening portions 621 to 627 has an annular shape and is formed to cover a region on the instep side and the sole side of the sock body 610.

The sock of the sixth embodiment is a female sock made in consideration of arch structure of a female foot portion. When a female wearer wears the sock, it is possible to more effectively correct the foot arch to a normal condition, and to correct the walking posture and the stationary posture to a normal condition.

### Modifications

Although the modes for carrying out the present invention have been described above with reference to specific examples, the present invention is not limited to the above-described embodiments, and modifications can be appropriately made within the scope of the gist of the present invention.

The sock according to the present invention only needs to cover at least the sole and the instep, and the sock according to the present invention includes socks of various lengths (short socks, long socks), tights, stockings, and the like. In addition, a portion or all of the toe may be exposed, or a portion covering the toe may be separated into several portions. From the viewpoint of preventing misalignment at the time of wearing, it is preferable that, in the sock, a portion covering the big toe and a portion covering the other toes are separated, a portion covering the big toe, a portion covering the second toe to the fourth toe, and a portion covering the little toe are separated, or portions covering all five toes are separated.

In the above embodiments, the tightening portions are formed in a band shape having substantially the same width in the circumferential direction, but the tightening portions may be formed to be wider on the big toe side than on the little toe side of the foot portion. By doing so, since the big toe side of the foot portion can be tightened in a wider area, the foot arch inclined toward the little toe side is effectively corrected.

In the above embodiment, the tightening portions are formed so as to cover one of the bone head portions or the basal portions of the metatarsals, but may be formed so as to cover both the bone head portions and the basal portions of the metatarsals and not to cover the portions between the bone head portions and the basal portions.

In addition, the tightening portion 220 of the sock 200 of the second embodiment may be formed such that the stretching force is lower in the direction indicated by the arrow A3 than in the direction indicated by the arrow A4. According to such structure, similarly to the sock 100 of the first embodiment, the tightening force of the tightening portion 220 becomes stronger on the big toe side, and a strong tightening force is applied to the big toe side of the foot portion. As a result, the tightening portion 220 generates a force that strongly twists the foot portion inward. As a result, the action of correcting the foot arch to a normal angle is more strongly exerted, and it is possible to obtain the action similar to the case where “inversion” of FIG. 16(a) or “adduction” of FIG. 16(e) is performed.

In the sock 200 of the second embodiment, the stretching direction is the same over the entire circumference in the instep side tightening portion 221 and the back side tightening portion 222, but the stretching direction may be switched by further dividing the instep side tightening portion 221 and the back side tightening portion 222 into a plurality of regions.

### Reference Example

A person's feet include a pivot foot (a foot for supporting the body) and a dominant foot (a foot for performing a motion). In the pivot foot, since the weight concentrates on the little toe side of the foot portion, the big toe side tends to be in a slightly lifted state. When the foot portion is deformed in this way, there is the problem that a load is applied to the biceps femoris, which is one of the muscles constituting the hamstring, so that the biceps femoris is enlarged, and the position of the pelvis connected to the biceps femoris is shifted.

The inventor of the present application has found that by wearing socks formed to tighten a specific toe in each of the pivot foot and the dominant foot, it is possible to prevent deformation of the feet due to deviation of the center of gravity and to prevent displacement of the pelvis. Hereinafter, the socks will be described as a reference example.

FIG. 15(a) is a view of the left foot on which the left foot sock is worn of socks 700 according to the reference example as viewed from the instep side, and FIG. 15(b) is a view of the right foot on which the right foot sock is worn of the socks 700 according to the reference example as viewed from the instep side. In this reference example, socks for a person whose left foot is a pivot foot and the right foot is a dominant foot will be described. Each of the socks 700 is a so-called five-toe type sock in which the portion covering the toe is separated into five portions, and includes a sock body 710 having a foot insertion portion 711 and a plurality of toe tightening portions 721, 722, 723, and 724 formed in the sock body 710. The toe tightening portions 721 and 722 are annularly formed so as to wind around a portion corresponding to a proximal interphalangeal joint C3a of the middle toe of the left foot and a portion corresponding to a proximal interphalangeal joint C3b of the little toe of the left foot, respectively. The toe tightening portions 723 and 724 are formed in a band shape so as to wind around a portion corresponding to a proximal interphalangeal joint C3c of the second toe of the right foot and a portion corresponding to a proximal interphalangeal joint C3d of the fourth toe of the right foot, respectively. The toe tightening portions 721 to 724 are made of a material such as a knitted fabric or a woven fabric having lower stretchability than other portions of the sock bodies 710. Therefore, the toe tightening portions 721 to 724 have a stronger tightening force than the other portions of the sock bodies 710.

The five toes are connected to various muscles for performing various movements of the foot portion (abduction/adduction, dorsiflexion/plantarflexion, supination/pronation, and inversion/eversion by combination of them, see FIG. 16). In the socks 700 according to this reference example, by tightening specific toes in the pivot foot and the dominant foot, muscles connected to the toes are stimulated to improve the function of the motion of the foot portion described above. Specifically, tightening the middle toe of the left foot (pivot foot) improves the function of adduction and abduction of the foot portion, and tightening the little toe of the left foot (pivot foot) improves the function of eversion of the foot portion. In addition, tightening the fourth toe of the right foot (dominant foot) improves the function of dorsiflexion of the foot portion, and tightening the second toe of the right foot (dominant foot) improves the function of inversion of the foot portion. By improving such various functions by the treatment, it is possible to resist deformation of the foot portion caused by deviation of the center of gravity of the wearer. In the socks for a person whose right foot is the pivot foot, the toe tightening portions 721 and 722 are formed in the sock for the right foot, and the toe tightening portions 723 and 724 are formed in the sock for the left foot (dominant foot).

### Modes

A person skilled in the art can understand that the previously described illustrative embodiments are specific examples of the following modes of the present invention.

(Clause 1) A sock according to one mode of the present invention includes: a sock body; and
an annular-shaped tightening portion provided in the sock body at a portion corresponding to at least one of bone head portions or basal portions of five metatarsals of a sock wearer so as to cover an instep and a sole,
wherein a tightening force of the tightening portion is larger than a tightening force of a portion other than the tightening portion of the sock body.

According to a sock according to Clause 1, the instep and the sole surrounding a metatarsophalangeal joint and/or a Lisfranc joint in a foot portion of the wearer of the sock are strongly tightened by the tightening portion, and a foot arch is corrected to a three-dimensional shape. In addition, since the metatarsophalangeal joint and/or the Lisfranc joint in the foot portion of the wearer are stimulated by the tightening force of the tightening portion, in a case where the foot arch of the wearer is inclined or the positions of the bones in the foot portion are shifted, it is possible to return them to a normal condition.

(Clause 2) A sock according to Clause 2 is the sock according to Clause 1, wherein
the tightening portion includes a medial tightening portion and a lateral tightening portion, and a tightening force of the medial tightening portion is larger than a tightening force of the lateral tightening portion.

(Clause 3) A sock according to Clause 3 is the sock according to Clause 1, wherein
the tightening portion is formed such that a stretch rate in directions inclined toward a tiptoe side from a little toe side to a big toe side is larger than a stretch rate in other directions.

According to the sock according to Clause 2 or Clause 3, since the tightening force of the tightening portion is shifted with respect to the direction in which the tightening portion annularly covers the instep and the sole, a tightening force with respect to the foot portion and a force that twists the foot are generated. In particular, in a case where the tightening portion is formed such that a stretch rate in directions inclined toward the tiptoe side from the little toe side to the big toe side is larger than the stretch rate in other directions, a force that twists the foot inward is generated. Therefore, by wearing the above sock, it is possible to correct the three-dimensional shape and inclination of the foot arch to an appropriate condition.

(Clause 4) A sock according to another mode of the present invention includes:
a sock body; and
an annular-shaped tightening portion provided in the sock body at a portion corresponding to at least one of a metatarsophalangeal joint, a Lisfranc joint, a Chopart joint, a toe joint, and a talocrural joint, which are joints in a foot portion of a wearer, wherein a tightening force of the tightening portion is larger than a tightening force of a portion other than the tightening portion of the sock body.

According to a sock according to Clause 4, the wearer of the sock is subjected to strong tightening by the tightening portion of the instep and the sole surrounding at least one of five types of joints in the foot portion, and a foot arch is corrected to a three-dimensional shape. In addition, since at least one of the five types of joints is stimulated by the tightening force of the tightening portion, in a case where the foot arch of the wearer is inclined or the positions of the bones in the foot portion are shifted, it is possible to return them to a normal condition.

(Clause 5) A sock according to Clause 5 is the sock according to Clause 4, wherein
the tightening portion is individually provided in the sock body at a portion corresponding to a joint of each of a big toe, a middle toe, and a little toe among five toes of the wearer.

According to the sock according to Clause 5, it is possible to obtain an effect particularly for a male wearer of correcting the three-dimensional shape of the foot arch to a correct condition.

(Clause 6) A sock according to Clause 6 is the sock according to Clause 4, wherein
the tightening portion is individually provided in the sock body at a portion corresponding to a joint of each of a big toe, a second toe, and a fourth toe among joints of five toes of the wearer.

According to the sock according to Clause 6, it is possible to obtain an effect particularly for a female wearer of correcting the three-dimensional shape of the foot arch to a correct condition.

(Clause 7) A sock according to Clause 7 is the sock according to Clause 5, wherein
the tightening portion is individually further provided in the sock body at a portion corresponding to each of the metatarsophalangeal joint, the Lisfranc joint, the Chopart joint, and the talocrural joint of the wearer.

(Clause 8) A sock according to Clause 8 is the sock according to Clause 6, wherein
the tightening portion is individually further provided in the sock body at a portion corresponding to each of the metatarsophalangeal joint, the Lisfranc joint, the Chopart joint, and the talocrural joint of the wearer.

The present inventor has found that the individual sites of the metatarsals to tighten are effective in improving certain types of bad posture. Specifically, in a bad posture in a condition where the lumbar vertebra is bent forward and the pelvis is tilted forward (hereinafter, referred to as “rounded back”), the weight is concentrated near the metatarsophalangeal joint. Therefore, especially by tightening the bone head portions of the metatarsals (the end portions on the metatarsophalangeal joint side), the structure of the foot arch is effectively corrected, and the rounded back is improved by spending time in the corrected condition. Similarly, in a bad posture in a condition where the lumbar vertebra is bent backward and the pelvis is tilted backward (hereinafter, referred to as “rounded lower back”), since the weight concentrates near the Lisfranc joint, tightening the basal portions of the metatarsals (end portions on the Lisfranc joint side) improves the rounded lower back.

A stronger surface pressure is applied to the portion on the big toe side of the instep and the sole in contact with the tightening portion than the portion on the little toe side. As a result, a force of tightening in the radial direction (tightening force) is applied to the portion corresponding to the tightening portion of the foot of the wearer, and the foot arch is corrected to a three-dimensional shape. In addition, in a case where the little toe side (lateral side) of the foot is inclined downward and the big toe side (medial side) is inclined upward due to the deviation of the center of gravity of the wearer and the big toe side is slightly lifted, the foot arch is corrected to an appropriate angle.

In a case where the metatarsals are tightened in a wide range, the correction according to the above certain types of bad posture cannot be performed. In addition, tightening unnecessary portions may deteriorate blood flow. Therefore, in order to effectively improve the above certain types of bad posture, it is preferable that the tightening portion is formed so as to cover either the bone head portions or the basal portions of the metatarsals, but not to cover the other.

In the above sock, the tightening portion is preferably wider on the big toe side than on the little toe side of the foot.

According to such structure, since the big toe side of the foot is tightened in a wider area, the correction effect of the foot arch is improved in a case where the little toe side (lateral side) of the foot is inclined downward and the big toe side (medial side) is inclined upward due to the deviation of the center of gravity of the wearer and the big toe side is slightly lifted.

(Clause 9) A sock according to Clause 9 is the sock according to any one of Clause 1 to Clause 8, wherein
one or more indications indicating a treatment site are provided on a surface of the sock body.

In the sock according to Clause 9, the treatment site refers to, for example, a site where the foot portion is twisted or pressed. The treatment includes applying finger pressure and massaging. The indication may be any mark as long as it serves as a mark of a treatment site, and a symbol, a character, a picture, a figure, or the like is included in the indication. In addition, the indication may be provided with a character indicating a treatment content, or an arrow symbol, a picture, or a figure indicating a direction in which force is applied in the case of applying the force to the treatment site.

According to the socks having the above structure, even a person who is unaccustomed to the treatment of the foot portion can perform appropriate treatment on an appropriate site of the foot portion of the wearer based on the indication on the socks.

### REFERENCE SIGNS LIST

100, 200, 300, 400, 500, 600 . . . Sock
110, 210, 310, 410, 510, 610 . . . Sock Body
120, 220, 320, 420, 520, 620 . . . Tightening Portion

1. A sock comprising:
a sock body; and
an annular-shaped first tightening portion provided in the sock body at a portion corresponding to at least one of bone head portions or basal portions of five metatarsals of a sock wearer so as to cover an instep and a sole of the sock wearer or at a portion corresponding to at least one of a metatarsophalangeal joint, a Lisfranc joint, a Chopart joint, a toe joint, and a talocrural joint, which are joints in a foot portion of the sock wearer,
wherein the first tightening portion includes a medial tightening portion and a lateral tightening portion, and a tightening force of the medial tightening portion is larger than a tightening force of the lateral tightening portion, and
wherein a tightening force of the medical tightening portion and a tightening force of the lateral tightening portion are larger than a tightening force of a portion other than the medical tightening portion and the lateral tightening portion of the sock body.

2-3. (canceled)

4. A sock comprising:
a sock body; and
an annular-shaped first tightening portion provided in the sock body at a portion corresponding to at least one of bone head portions or basal portions of five metatarsals of a sock wearer so as to cover an instep and a sole of the sock wearer or at a portion corresponding to at least one of a metatarsophalangeal joint, a Lisfranc joint, a Chopart joint, a toe joint, and a talocrural joint, which are joints in a foot portion of a wearer,
wherein the first tightening portion is formed such that a stretch rate in directions inclined toward a tiptoe side from a little toe side to a big toe side is larger than a stretch rate in other directions.

5. The sock according to claim 1, wherein an annular-shaped second tightening portion is individually provided in the sock body at a portion corresponding to a joint of each of a big toe, a middle toe, and a little toe among five toes of the sock wearer and wherein a tightening force of the second tightening portion is larger than a tightening force of a portion other than the first tightening portion and the second tightening portion of the sock body.

6. The sock according to claim 1, wherein an annular-shaped third tightening portion is individually provided in the sock body at a portion corresponding to a joint of each of a big toe, a second toe, and a fourth toe among joints of five toes of the sock wearer.

7. The sock according to claim 5, wherein the first tightening portion is individually provided in the sock body at a portion corresponding to each of the metatarsophalangeal joint, the Lisfranc joint, the Chopart joint, and the talocrural joint of the sock wearer.

8. The sock according to claim 6, wherein the first tightening portion is individually provided in the sock body at a portion corresponding to each of the metatarsophalangeal joint, the Lisfranc joint, the Chopart joint, and the talocrural joint of the sock wearer.

9. The sock according to claim 1, wherein one or more indications indicating a treatment site are provided on a surface of the sock body.

10. The sock according to claim 1, wherein the first tightening portion provided in the sock body at a portion corresponding to the talocrural joint of the sock wearer.

11. The sock according to claim 4, wherein the first tightening portion provided in the sock body at a portion corresponding to the talocrural joint of the sock wearer.

12. The sock according to claim 4, wherein an annular-shaped second tightening portion is individually provided in the sock body at a portion corresponding to a joint of each of a big toe, a middle toe, and a little toe among five toes of the sock wearer and wherein a tightening force of the second tightening portion is larger than a tightening force of a portion other than the first tightening portion and the second tightening portion of the sock body.

13. The sock according to claim 4, wherein an annular-shaped third tightening portion is individually provided in the sock body at a portion corresponding to a joint of each of a big toe, a second toe, and a fourth toe among joints of five toes of the sock wearer.

14. The sock according to claim 12, wherein the first tightening portion is individually provided in the sock body at a portion corresponding to each of the metatarsophalangeal joint, the Lisfranc joint, the Chopart joint, and the talocrural joint of the sock wearer.

15. The sock according to claim 13, wherein the first tightening portion is individually provided in the sock body at a portion corresponding to each of the metatarsophalangeal joint, the Lisfranc joint, the Chopart joint, and the talocrural joint of the sock wearer.

16. The sock according to claim 4, wherein one or more indications indicating a treatment site are provided on a surface of the sock body.
